(19) United States
(12) Patent Application Publication
(10) Pub. No.: US 2017/0120004 A1
(43) Pub. Date: May 4, 2017

(54) CATHETER NAVIGATION SYSTEM

(71) Applicant: GSM KOREA CO.,LTD., Gwangju (KR)

(72) Inventor: Jung-Won SHIN, Gwangju (KR)

(21) Appl. No.: 14/958,314

(22) Filed: Dec. 3, 2015

(30) Foreign Application Priority Data
Nov. 2, 2015 (KR) 10-2015-0153084

(51) Int. Cl. A61M 25/01; A61B 5/00; A61B 1/00; A61B 34/20; A61B 90/00

(52) U.S. Cl. CPC A61M 25/0147; A61M 25/0136; A61B 34/20; A61B 90/36; A61B 1/00165; A61B 5/0084; A61M 2230/30; A61M 2025/015

(57) ABSTRACT

A catheter navigation system includes a handle (100) which is operated by a user; a tube (200) which is configured to be connected to the handle (100) and is inserted into a human body; a tube navigation unit (300) which pushes one side of the tube (200) while pulling the other side so as to bend the tube (200) and thus navigate a direction of the tube; a plastic optical fiber (400) which is built in the tube (200) to transmit a light, with one end of the plastic optical fiber being exposed from a distal end of the tube (200); and a cap (500) which is connected to the distal end of the tube (200) to close the distal end of the tube (200).

### CROSS REFERENCE

This application claims foreign priority under Paris Convention to Korean Patent Application No. 10-2015-0153084, filed 2 Nov. 2015, with the Korean Intellectual Property Office.

### BACKGROUND

The present invention relates to a catheter navigation system capable of more accurately navigating and positioning a tube to be inserted through a patient's body to a surgery site or a region of interest.

In general, a catheter is a medical appliance of a tube type which is configured to be inserted into a body cavity or an organ of a patient at a surgical operation, and is referred to as a probe. The catheter can be made of various materials, sizes, and shapes, depending upon its application. The catheter is made of rubber, plastic or metal. The catheter is widely used to excrete contents from various body organs, such as a body cavity or an organ, introduce drugs or cleaning solutions into the body organs, and/or inspect the inside of the body organs.

The conventional catheter is largely divided into two catheters, that is, a drug introducing catheter, of which a drug is able to introduce into a tube, and an inspection catheter, of which a plastic optical fiber is provided in the tube to take a picture of the inside of the body organ. The conventional catheter has a problem in that since the tube is very thin and long, drug introduction and inspection cannot be simultaneously carried out through one tube. Therefore, a drug introducing catheter and an inspection catheter should be separately provided, and two catheters should be alternatively used in order, which gives inconvenience to a surgeon and delays the operation time. Meanwhile, there is another problem in that if the drug introduction and the inspection can be carried out through one tube, the size of the tube is increased to magnify the surgery site, and manipulability of the catheter is decreased to lower the accuracy of the operation.

The background related to the present invention is disclosed by Korean Patent No. 10-1219710, entitled “probe and device for detecting abnormality of intervertebral disc.”

### SUMMARY OF THE INVENTION

Therefore, the present invention has been made in view of the above problems, and an object of the present invention is to provide a catheter navigation system capable of more accurately navigating and positioning a tube to be inserted through a patient's body to a surgery site or a region of interest.

According to one aspect of the present invention, there is provided a catheter navigation system including: a handle which is operated by a user; a tube which is configured to be connected to the handle and is inserted into a human body; a tube navigation unit which pushes one side of the tube while pulling the other side so as to bend the tube and thus navigate a direction of the tube; a plastic optical fiber which is built in the tube to transmit a light, with one end of the plastic optical fiber being exposed from a distal end of the tube; and a cap which is connected to the distal end of the tube to close the distal end of the tube.

Preferably, the tube navigation unit includes wires which are housed parallel to each other in the tube, with one ends of the wires being fixed to the distal end of the tube, and the other ends being respectively positioned in the handle, and a wire operating unit which is connected to the other ends of the wires so as to pull one wire and push the other wire.

Preferably, the wire operating unit includes an operation handle which is connected to the other ends of the wires so as to pull or push one wire and push or pull the opposite wire, and a rotation support member which is provided to the handle so as to support swing movement of the operation handle.

Preferably, the wires are distributed and built in the tube in four directions, in which the end of the respective wires is fixed to the distal end of the tube, while the other end is connected to the operation handle in four directions, and the operation handle has a ring-shaped body which is operated by the user, and protrusions which protrude inwardly from the body in four directions to fix the other ends of the wires, respectively.

Preferably, the rotation support member has a female-type rotation support portion for fixing the protrusions, and a male-type rotation portion to which the female-type support portion is connected to be able to rotate in four directions.

Preferably, the plastic optical fiber includes an optic angle enlarging portion for condensing an incident light so as to enlarge an angle of the light to be emitted, and the optic angle enlarging portion is connected to an inlet side of the plastic optical fiber, and is formed in a funnel shape, of which an inlet for receiving a light illuminated from a light source is formed to be wider than an outlet for emitting the light.

Preferably, the catheter navigation system further includes a drug supply unit which is built in the tube to supply a drug into an inside of a human body. The drug supply unit includes a drug feed tube which is built in the tube to discharge the drug to be fed from the exterior through a distal end thereof, a drug pump for pumping and feeding the drug to the drug feed tube, a body status measuring member for measuring a status of the human body, and a pump operating member which compares the status of the human body measured by the body status measuring member and a preset reference value, and controls operating speed of the drum pump to adjust a supply amount of the drug according to the compared result.

Preferably, the body status measuring member has a blood pressure sensor which is provided to the distal end of the tube to be close to the drug feed tube, so as to measure blood pressure of blood flowing in front of the distal end of the tube inserted into the human body.

Preferably, the catheter navigation system further includes a distance measuring member for measuring a distance of a subject through the plastic optical fiber; a grid frame having a plurality of horizontal lines and a plurality of vertical lines which are spaced at regular intervals, and are displayed on a screen of a monitor; and a dimension calculating member for measuring a dimension of the subject by use of the distance measured by the distance measuring member and the grid frame. The dimension calculating member has a grid recognizing part for recognizing the horizontal lines and the vertical lines which are close to an outside of the subject enclosed by the grid frame, a calculation part for calculating an interval of the horizontal lines recognized by the grid recognizing part as a horizontal value, and calculating an interval of the vertical lines as a vertical value, and a value adjusting part for decreasing the horizontal value and the vertical value which are calculated by the calculation part as much as the increased distance value measured by the distance measuring member, or increasing the horizontal value and the vertical value which are calculated by the calculation part as much as the decreased distance value measured by the distance measuring member.

With the above configuration of the catheter navigation system of the present invention, the tube to be inserted into the human body can be accurately navigated and positioned at a surgery site or an inspection position, thereby accurately carrying out the operation or the inspection

### BRIEF DESCRIPTION OF THE DRAWINGS

FIG. 1 is a view schematically illustrating a catheter navigation system according to one embodiment of the present invention.

FIG. 2 is a cross-sectional view illustrating the inside of the catheter navigation system according to the embodiment of the present invention.

FIG. 3 is a view illustrating a tube navigation unit for manipulating a direction of a tube according to the embodiment of the present invention.

FIG. 4 is a partially perspective view of major parts of the catheter navigation system according to one embodiment of the present invention.

FIGS. 5A to 5C are view illustrating samples of an optic angle enlarging portion applied to a plastic optical fiber according to the embodiment of the present invention.

FIGS. 6A and 6B are views illustrating a process of measuring the size of a tissue according to the present invention.

FIGS. 7A and 7B are views the state of the tissue displayed on a monitor according to the present invention.

FIG. 8 is a block diagram schematically illustrating major parts of the catheter navigation system according to the embodiment of the present invention.

### DETAILED DESCRIPTION OF THE PREFERRED EMBODIMENT

Hereinafter, a catheter navigation system according to one embodiment of the present invention will be described in detail with reference to the accompanying drawings.

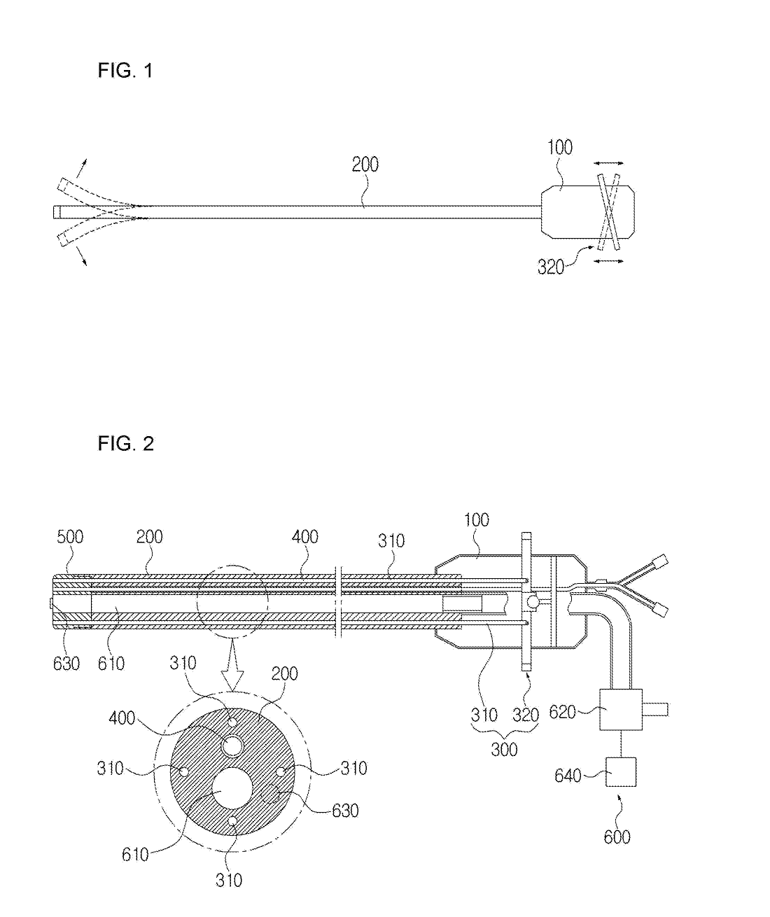

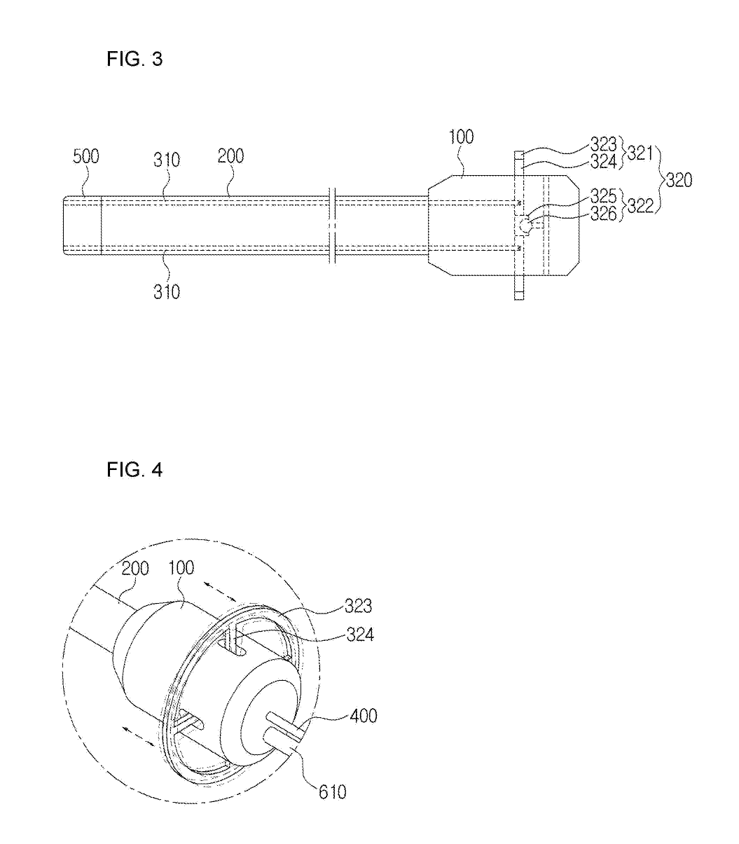

As illustrated in FIGS. 1 to 5, the catheter navigation system of the present invention includes a handle 100, a tube 200, a tube navigation unit 300, a plastic optical fiber 400, and a cap 500.

The handle 100 is held by a user, and is formed in a case shape to form a space therein, in which components are installed.

The tube 200 is configured to be connected to the handle 100 and inserted into a human body. The tube 200 is connected to the handle 100 at one end thereof in a communication manner, and has a plurality of channels with both ends being opened, through which wires 310 of the tube navigation unit 300, the plastic optical fiber 500, and drugs and inspection appliances pass. The tube 200 is formed in an elongated shape so as to be easily inserted into the body cavity. Preferably, the tube has a diameter of about 2.5 mm. The channels are passages formed in a longitudinal direction of the tube 200.

The tube navigation unit 300 includes the wires which are housed parallel to each other in the tube, the wires pushing one side of the tube 200 while pulling the other side of the tube so as to navigate and position the tube 200 by bending an end of the tube 200, with one ends of the wires being fixed to the distal end of the tube 200, and the other ends being respectively positioned in the handle 100, and a wire operating unit 320 which is connected to the other ends of the wires 310 so as to pull one wire 310 and push the other wire 310.

The wires 310 are built in the channels of the tube 200, and push the end of the tube if the wire is pushed by an external force, or pull the end of the tube if the wire is pulled by the external force. The wires 310 may be made of a soft cord which is flexible by the external force, like a synthetic resin. Accordingly, if the wire 310 positioned at an upper portion of the tube 200 is pulled, the wire 310 positioned at a lower portion of the tube 200 is pushed, such that the distal end of the tube 200 is bent in an upward direction.

The wire operating unit 320 includes an operation handle 321 which is connected to the other ends of the wires 310 so as to pull or push one wire 310 and push or pull the opposite wire 310, and a rotation support member 322 which is provided to the handle 100 so as to support swing movement of the operation handle 321.

The wires 310 are distributed and built in the tube 200 in four directions, in which the end of the respective wires 310 is fixed to the distal end of the tube 200, while the other end is connected to the operation handle 321 in four directions.

The operation handle 321 has a ring-shaped body 323 which is operated by the user, and protrusions 324 protruding inwardly from the body 323 in four directions to fix the other ends of the wires 310.

The rotation support member 322 has a female-type rotation support portion 325 for fixing the protrusions 324, and a male-type rotation portion 326 to which the female-type support portion 325 is connected to be able to rotate in four directions.

The body 323 is formed in a ring shape, and is positioned on the outside of the handle 10.

The protrusions 324 protrude inwardly from the body 323 in a substantially cross shape, and are fixed to the female-type rotation support portion 325 of the rotation support member 322 while penetrating the inside of handle 10.

The female-type support portion 325 is formed with a substantially dome-shaped groove on an inner portion thereof, and the male-type rotation support portion 326 is formed with a spherical boss on a distal end thereof. Thus, the female support portion 325 can be rotated around the male-type rotation support portion 326 in the four directions. The rotation support member 322 may be formed of a ball joint.

If the one side of the operation handle 321 is pulled rearward by the user, the opposite side of the operation handle 321 which can swing on the rotation support member 322 is moved forward. That is, the operation handle 321 swings around the rotation support member 322 as an axis. In this instance, the wire 310 connected to the operation handle 321 moving rearward is also pulled to the rearward. On the contrary, the wire 310 connected to the operation handle 321 moving forward is also pushed to the forward. Thus, the distal end of the tube 200 is bent toward the pulling direction of the wire 310.

With the above configuration, the wires 310 can be moved in opposite directions by use of one operation handle 321, thereby quickly bending the tube 200.

If the wires 310 made of two, four, eight, or 16 cords are disposed in the tube 200 at regular intervals and are connected to the operation handle 321, the tube 200 can be precisely bent in two, four, eight or 16 directions.

The plastic optical fiber (POF) 400 is to transmit the light in a state in which the plastic optical fiber is built in the tube 200, with one end being exposed from the distal end of the tube 200. Illumination light can be transmitted through the plastic optical fiber, and a camera can be connected to the plastic optical fiber to take a picture. The plastic optical fiber 400 may be built in other channel of the tube 200, and the end of the plastic optical fiber may be exposed outwardly from the distal end of the tube 200, so that the inside of the human body can be taken by the camera connected to the plastic optical fiber 400, and the illumination light can be provided onto the human body which is taken by the camera, by illumination connected to the plastic optical fiber 400.

Also, the plastic optical fiber 400 has an optic angle enlarging portion 430 for condensing the incident light so as to enlarge an angle of the light emitted.

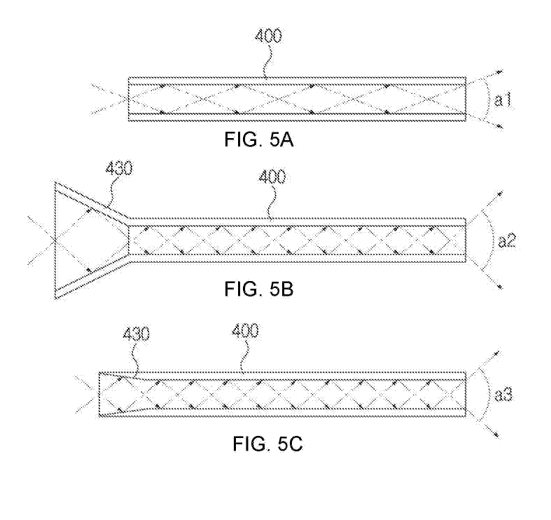

The optic angle enlarging portion 430 is connected to an inlet side of the plastic optical fiber 400, and is formed in a coupler of a funnel shape, of which an inlet for receiving the light is formed to be wider than an outlet for emitting the light, as illustrated in FIG. 5B. Also, the optic angle enlarging portion 430 may be formed in such a way that an inner peripheral surface of the inlet side of the plastic optical fiber 400 is enlarged than an inner peripheral surface of the outlet side, as illustrated in FIG. 5C.

As illustrated in FIG. 5A, a light of a substantially straight shape having a narrow incident angle is input into an inlet side of a plastic optical fiber 420 for illumination, and then a light of a straight shape can be outputted from the outlet side as much as the incident light. That is, the light can be outputted to have a narrow optic angle a1.

As illustrated in FIG. 5B, however, if the light is condensed by the coupler, that is, the optic angle enlarging portion 430, and then is inputted into the inlet side of the plastic optic fiber 400, the light can be outputted at an enlarged optic angle from the output side as much as the condensed light. For example, if the light is incident and condensed at an angle of about 120 degrees by a slope surface formed on the inner portion of the coupler, that is, the optic angle enlarging portion 430, and then is inputted into the inlet side of the plastic optical fiber 400, the light having an optic angle a2 which corresponds to about 120 degrees can be outputted from the outlet side of the plastic optical fiber 400.

As illustrated in FIG. 5C, in the case where the optic angle enlarging portion 430 has the slope surface which is formed on the inner peripheral surface of the inlet side of the plastic optical fiber 400 to have an increased angle, the light having the increased optic angle a3 can be outputted.

As described above, if the plastic optical fiber 400 has the plastic optical fiber 420 for illumination, and the optic angle enlarging portion 430, the illumination of the enlarged angle can be applied to the bodily tissue of a wider range. If the plastic optical fiber 400 has the optic angle enlarging portion 430, the angle of view is increased, thereby providing the illumination in the wider range, as well as taking a picture of the wider bodily tissue.

The cap 500 is engaged to the distal end of the tube 200, and is formed with mounting holes corresponding to the channels of the tube 200 to receive the wires 310 and the plastic optical fiber 400 therein.

The plastic optical fiber 400 is provided to be exposed from the front surface of the cap 500, and the front surface of the cap 500 and the front surface of the plastic optical fiber 400 may be polished to have a curved surface having a desired curvature which protrudes forward.

Since the cap 500 is mounted to the distal end of the tube 200, and the wire 310 is fixed to the cap 500, the wires can be firmly fixed, thereby preventing the ends of the wires 310 from being separated or broken due to repeated use and thus improving its durability.

In addition, the wires 310 and the plastic optical fiber 400 are not directly coupled to the tube 200, but are fixed to the cap 500 at the distal end of the tube 200, thereby firmly maintaining the fixed state.

The operation of the catheter navigation system according to the present invention will now be described.

First, after the tube 200 is inserted into the human body, the user operates the wire operating unit 320 to navigate the distal end of the tube 200 in a desired direction.

The user swings the operation handle 321 of the wire operating unit 320 to allow the tube 200 to face in any one direction. Specifically, the distal end of the tube 200 can be accurately navigated and positioned at a desired position by the simple operation of the user, thereby improving the accuracy of the operation or inspection.

Therefore, the user can take a picture of the inside of the human body by the plastic optical fiber 400 in the state in which the distal end of the tube 200 is positioned at the desired position.

Also, the catheter navigation system of the present invention includes a drug supply unit 600 which is built in the tube 200 to supply a drug into the inside of the human body.

The drug supply unit 600 includes a drug feed tube 610 which is built in the tube 200 to discharge the drug to be fed from the exterior through a distal end thereof, a drug pump 620 for pumping and feeding the drug to the drug feed tube 610, a body status measuring member 630 for measuring the status of the human body, and a pump operating member 640 which compares the status of the human body measured by the body status measuring member 630 and a preset reference value, and controls the operating speed of the drum pump 620 to adjust a supply amount of the drug according to the compared result.

The drug feed tube 610 is built in the other channel of the tube 200 to guide the drug fed from the exterior to the inside of the human body. In this instance, the drug may be a saline solution for washing the tissue of the human body to increase the visibility, or a remedy drug for treating the tissue of the human body.

The drug pump 620 can forcibly feed the prepared drug to the drug feed tube 610.

The body status measuring member 630 may be a blood pressure sensor which is provided to the distal end of the tube 200 to be close to the drug feed tube 610, so as to measure blood pressure of blood flowing in front of the distal end of the tube 200 inserted into the human body. Accordingly, the internal pressure of the human body can be measured by the body status measuring member 630.

The pump operating member 640 may include a controller which can be operated by the user so as to allow the drug pump 620 to feed the drug. A reference value can be set to be slightly lower than the normal blood pressure measured in the human body of the patient, and can be arbitrarily set depending upon the status of the patient.

Accordingly, if the drug pump 620 is operated in the state in which the tube 200 is inserted into the inside of the human body, the drug is sprayed from the distal end of the tube 200 through the drug feed tube 610, and thus the tissue of the human body positioned at the distal end of the tube 200 is washed, thereby securing the visibility of the plastic optical fiber 400 exposed from the distal end of the tube 200. In this instance, since the blood pressure of the human body is measured by the body status measuring member 630, the drug is sprayed at pressure lower than the blood pressure, on the basis of the measured blood pressure. Specifically, it is possible to wash the tissue of the human body, while minimizing the increase in internal pressure of the human body due to the drug to be sprayed.

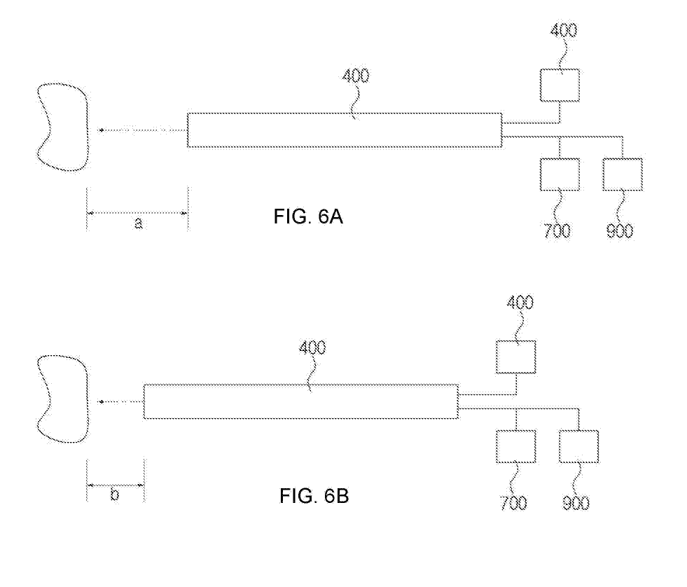

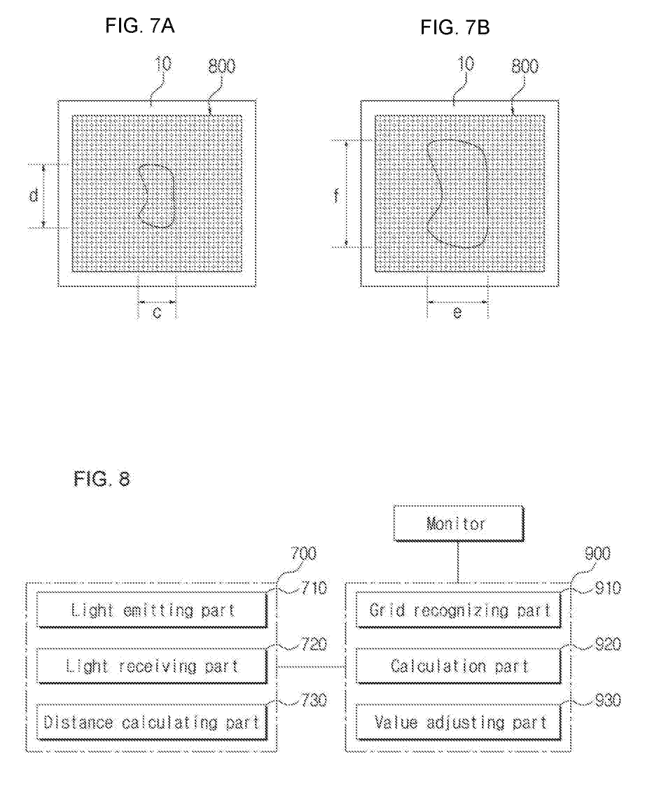

As illustrated in FIGS. 6 to 8, the catheter navigation system of the present invention includes a distance measuring member 700 for measuring a distance of a subject through the plastic optical fiber 400, a grid frame 800 having a plurality of horizontal lines and a plurality of vertical lines which are spaced at regular intervals, and displayed on a screen of a monitor 10, and a dimension calculating member 900 for measuring a dimension of the subject by use of the distance measured by the distance measuring member 700 and the grid frame 800.

The distance measuring member 700 has a light emitting part 710 for emitting a light to the plastic optical fiber 400, a light receiving part 720 for receiving the light emitted from the light emitting part 710, irradiated onto the subject through the plastic optical fiber 400, and then reflected from the subject, and a distance calculating part 730 for calculating a distance from the subject to the distal end of the plastic optical fiber 400 on the basis of the distance from the subject to the light receiving part 720 and a length of the plastic optical fiber 400 according to a light receiving state of the light receiving part 720.

The distance calculating part 730 calculates the distance from the subject to the light receiving part 720, and calculates a distance value from the subject to the distal end of the plastic optical fiber 400 from the calculated distance, except for the distance the length of the plastic optical fiber 400.

The grid frame 800 may form the vertical lines and the horizontal lines on the screen of the monitor 10 in the shape of a checkerboard. In this instance, the interval of the vertical lines or the horizontal lines can be set by about 10 to 100 nanometers so as to accurately measure the size of a microscale tissue.

The dimension calculating member 900 has a grid recognizing part 910 for recognizing the horizontal lines and the vertical lines which are close to the outside of the subject enclosed by the grid frame 800, a calculation part 920 for calculating the interval of the horizontal lines recognized by the grid recognizing part 910 as a horizontal value, and calculating an interval of the vertical lines as a vertical value, and a value adjusting part 930 for decreasing the horizontal value and the vertical value which are calculated by the calculation part 920 as much as the increased distance value measured by the distance measuring member 700, or increasing the horizontal value and the vertical value which are calculated by the calculation part 920 as much as the decreased distance value measured by the distance measuring member 700.

The grid recognizing part 910 recognizes the horizontal lines and the vertical lines which are positioned at the outermost periphery of the tissue of the subject.

The calculation part 920 calculates the interval of the horizontal lines and the interval of the vertical lines, such that the user can see the whole area.

Since the value adjusting part 930 is configured to adjust the horizontal value and the vertical value according to the increase or decrease in distance to be measured, the user can see the size of the tissue corresponding to the actual size. The value adjusting part 930 may be configured so that the horizontal value and the vertical value are displayed as numerals on the screen of the monitor 10.

Accordingly, the interval c of the horizontal lines and the interval d of the vertical lines which are positioned at the periphery of the tissue displayed on the monitor 10 are decreased at the distance of the distal end of the plastic optical fiber 400 which is far away from the tissue of the subject. On the contrary, the interval e of the horizontal lines and the interval f of the vertical lines which are positioned at the periphery of the tissue displayed on the monitor 10 are increased at the distance of the distal end of the plastic optical fiber 400 which is close to the tissue of the subject. Therefore, the user can see the actual correct size of the tissue through the monitor 10, which can be of help to the tissue inspection or treatment.

With the above configuration, since the drug introduction and the photographing can be carried out by use of one tube, the catheter is easy to use, and the inspection time can be shortened. Therefore, the present invention can be widely applied to the catheter.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A catheter navigation system comprising:
a handle which is operated by a user;
a tube which is configured to be connected to the handle and is inserted into a human body;
a tube navigation unit which pushes one side of the tube while pulling the other side so as to bend the tube and thus navigate a direction of the tube;
a plastic optical fiber which is built in the tube to transmit a light, with one end of the plastic optical fiber being exposed from a distal end of the tube; and
a cap which is connected to the distal end of the tube to close the distal end of the tube.

2. The catheter navigation system according to claim 1, wherein the tube navigation unit includes wires which are housed parallel to each other in the tube, with one ends of the wires being fixed to the distal end of the tube, and the other ends being respectively positioned in the handle, and a wire operating unit which is connected to the other ends of the wires so as to pull one wire and push the other wire.

3. The catheter navigation system according to claim 2, wherein the wire operating unit includes an operation handle which is connected to the other ends of the wires so as to pull or push one wire and push or pull the opposite wire, and a rotation support member which is provided to the handle so as to support swing movement of the operation handle.

4. The catheter navigation system according to claim 3, wherein the wires are distributed and built in the tube in four directions, in which the end of the respective wires is fixed to the distal end of the tube, while the other end is connected to the operation handle in four directions, and
the operation handle has a ring-shaped body which is operated by the user, and protrusions which protrude inwardly from the body in four directions to fix the other ends of the wires, respectively.

5. The catheter navigation system according to claim 3, wherein the rotation support member has a female-type rotation support portion for fixing the protrusions, and a male-type rotation portion to which the female-type support portion is connected to be able to rotate in four directions.

6. The catheter navigation system according to claim 1, wherein the plastic optical fiber includes an optic angle enlarging portion for condensing an incident light so as to enlarge an angle of the light to be emitted, and
the optic angle enlarging portion is connected to an inlet side of the plastic optical fiber, and is formed in a funnel shape, of which an inlet for receiving a light illuminated from a light source is formed to be wider than an outlet for emitting the light.

7. The catheter navigation system according to claim 1, further comprising a drug supply unit which is built in the tube to supply a drug into an inside of a human body, and
the drug supply unit including a drug feed tube which is built in the tube to discharge the drug to be fed from the exterior through a distal end thereof, a drug pump for pumping and feeding the drug to the drug feed tube, a body status measuring member for measuring a status of the human body, and a pump operating member which compares the status of the human body measured by the body status measuring member and a preset reference value, and controls operating speed of the drum pump to adjust a supply amount of the drug according to the compared result.

8. The catheter navigation system according to claim 7, wherein the body status measuring member has a blood pressure sensor which is provided to the distal end of the tube to be close to the drug feed tube, so as to measure blood pressure of blood flowing in front of the distal end of the tube inserted into the human body.

9. The catheter navigation system according to claim 1, further comprising
a distance measuring member for measuring a distance of a subject through the plastic optical fiber;
a grid frame having a plurality of horizontal lines and a plurality of vertical lines which are spaced at regular intervals, and are displayed on a screen of a monitor; and
a dimension calculating member for measuring a dimension of the subject by use of the distance measured by the distance measuring member and the grid frame,
the dimension calculating member having a grid recognizing part for recognizing the horizontal lines and the vertical lines which are close to an outside of the subject enclosed by the grid frame, a calculation part for calculating an interval of the horizontal lines recognized by the grid recognizing part as a horizontal value, and calculating an interval of the vertical lines as a vertical value, and a value adjusting part for decreasing the horizontal value and the vertical value which are calculated by the calculation part as much as the increased distance value measured by the distance measuring member, or increasing the horizontal value and the vertical value which are calculated by the calculation part as much as the decreased distance value measured by the distance measuring member.
